SEQUENCE LISTING (3 sequences)

SEQ ID NO: 1 - protein, 418 aa, Homo sapiens
MPSSVSWGILLLAGLCCLVPVSLAEDPQGDAAQKTDTSHHDQDHPTFNKITPNLAEFAFSLYRQLAHQSNSTNIFFSPVSIATAFAMLSLGTKADTHDEILEGLNFNLTEIPEAQIHEGFQELLRTLNQPDSQLQLTTGNGLFLSEGLKLVDKFLEDVKKLYHSEAFTVNFGDTEEAKKQINDYVEKGTQGKIVDLVKELDRDTVFALVNYIFFKGKWERPFEVKDTEEEDFHVDQVTTVKVPMMKRLGMFNIQHCKKLSSWVLLMKYLGNATAIFFLPDEGKLQHLENELTHDIITKFLENEDRRSASLHLPKLSITGTYDLKSVLGQLGITKVFSNGADLSGVTEEAPLKLSKAVHKAVLTIDEKGTEAAGAMFLEAIPMSIPPEVKFNKPFVFLMIEQNTKSPLFMGKVVNPTQK

SEQ ID NO: 2 - DNA, 3006 bp, Homo sapiens
  source 1..3006: mol_type = genomic DNA
ctcctcagcttcaggcaccaccactgacctgggacagtgaatcgacaatgccgtcttctgtctcgtggggcatcctcctgctggcaggcctgtgctgcctggtccctgtctccctggctgaggatccccagggagatgctgcccagaagacagatacatcccaccatgatcaggatcacccaaccttcaacaagatcacccccaacctggctgagttcgccttcagcctataccgccagctggcacaccagtccaacagcaccaatatcttcttctccccagtgagcatcgctacagcctttgcaatgctctccctggggaccaaggctgacactcacgatgaaatcctggagggcctgaatttcaacctcacggagattccggaggctcagatccatgaaggcttccaggaactcctccgtaccctcaaccagccagacagccagctccagctgaccaccggcaatggcctgttcctcagcgagggcctgaagctagtggataagtttttggaggatgttaaaaagttgtaccactcagaagccttcactgtcaacttcggggacaccgaagaggccaagaaacagatcaacgattacgtggagaagggtactcaagggaaaattgtggatttggtcaaggagcttgacagagacacagtttttgctctggtgaattacatcttctttaaaggcaaatgggagagaccctttgaagtcaaggacaccgaggaagaggacttccacgtggaccaggtgaccaccgtgaaggtgcctatgatgaagcgtttaggcatgtttaacatccagcactgtaagaagctgtccagctgggtgctgctgatgaaatacctgggcaatgccaccgccatcttcttcctgcctgatgaggggaaactacagcacctggaaaatgaactcacccacgatatcatcaccaagttcctggaaaatgaagacagaaggtctgccagcttacatttacccaaactgtccattactggaacctatgatctgaagagcgtcctgggtcaactgggcatcactaaggtcttcagcaatggggctgacctctccggggtcacagaggaggcacccctgaagctctccaaggccgtgcataaggctgtgctgaccatcgacgagaaagggactgaagctgctggggccatgtttttagaggccatacccatgtctatcccccccgaggtcaagttcaacaaaccctttgtcttcttaatgattgaacaaaataccaagtctcccctcttcatgggaaaagtggtgaatcccacccaaaaataactgcctctcgctcctcaacccctcccctccatccctggccccctccctggatgacattaaagaagggttgagctggtccctgcctgcatgtgactgtaaatccctcccatgttttctctgagtctccctttgcctgctgaggctgtatgtgggctccaggtaacagtgctgtcttcgggccccctgaactgtgttcatggagcatctggctgggtaggcacatgctgggcttgaatccaggggggactgaatcctcagcttacggacctgggcccatctgtttctggagggctccagtcttccttgtcctgtcttggagtccccaagaaggaatcacaggggaggaaccagataccagccatgaccccaggctccaccaagcatcttcatgtccccctgctcatcccccactcccccccacccagagttgctcatcctgccagggctggctgtgcccaccccaaggctgccctcctgggggccccagaactgcctgatcgtgccgtggcccagttttgtggcatctgcagcaacacaagagagaggacaatgtcctcctcttgacccgctgtcacctaaccagactcgggccctgcacctctcaggcacttctggaaaatgactgaggcagattcttcctgaagcccattctccatggggcaacaaggacacctattctgtccttgtccttccatcgctgccccagaaagcctcacatatctccgtttagaatcaggtcccttctccccagatgaagaggagggtctctgctttgttttctctatctcctcctcagacttgaccaggcccagcaggccccagaagaccattaccctatatcccttctcctccctagtcacatggccataggcctgctgatggctcaggaaggccattgcaaggactcctcagctatgggagaggaagcacatcacccattgacccccgcaacccctccctttcctcctctgagtcccgactggggccacatgcagcctgacttctttgtgcctgttgctgtccctgcagtcttcagagggccaccgcagctccagtgccacggcaggaggctgttcctgaatagcccctgtggtaagggccaggagagtccttccatcctccaaggccctgctaaaggacacagcagccaggaagtcccctgggcccctagctgaaggacagcctgctccctccgtctctaccaggaatggccttgtcctatggaaggcactgccccatcccaaactaatctaggaatcactgtctaaccactcactgtcatgaatgtgtacttaaaggatgaggttgagtcataccaaatagtgatttcgatagttcaaaatggtgaaattagcaattctacatgattcagtctaatcaatggataccgactgtttcccacacaagtctcctgttctcttaagcttactcactgacagcctttcactctccacaaatacattaaagatatggccatcaccaagccccctaggatgacaccagacctgagagtctgaagacctggatccaagttctgacttttccccctgacagctgtgtgaccttcgtgaagtcgccaaacctctctgagccccagtcattgctagtaagacctgcctttgagttggtatgatgttcaagttagataacaaaatgtttatacccattagaacagagaataaatagaactacatttcttgca

SEQ ID NO: 3 - DNA, 3475 bp, Homo sapiens
  source 1..3475: mol_type = genomic DNA
agagtcctgagctgaaccaagaaggaggagggggtcgggcctccgaggaaggcctagccgctgctgctgccaggaattccaggttggaggggcggcaacctcctgccagccttcaggccactctcctgtgcctgccagaagagacagagcttgaggagagcttgaggagagcaggaaaggtgggacattgctgctgctgctcactcagttccacagggcggcagtaagtcttcagcatcaggcattttggggtgactcagtaaatggtagatcttgctaccagtggaacagccactaaggattctgcagtgagagcagagggccagctaagtggtactctcccagagactgtctgactcacgccaccccctccaccttggacacaggacgctgtggtttctgagccagcagcctcccccgttgcccctctggatccactgcttaaatacggacgaggacagggccctgtctcctcagcttcaggcaccaccactgacctgggacagtgaatcgacaatgccgtcttctgtctcgtggggcatcctcctgctggcaggcctgtgctgcctggtccctgtctccctggctgaggatccccagggagatgctgcccagaagacagatacatcccaccatgatcaggatcacccaaccttcaacaagatcacccccaacctggctgagttcgccttcagcctataccgccagctggcacaccagtccaacagcaccaatatcttcttctccccagtgagcatcgctacagcctttgcaatgctctccctggggaccaaggctgacactcacgatgaaatcctggagggcctgaatttcaacctcacggagattccggaggctcagatccatgaaggcttccaggaactcctccgtaccctcaaccagccagacagccagctccagctgaccaccggcaatggcctgttcctcagcgagggcctgaagctagtggataagtttttggaggatgttaaaaagttgtaccactcagaagccttcactgtcaacttcggggacaccgaagaggccaagaaacagatcaacgattacgtggagaagggtactcaagggaaaattgtggatttggtcaaggagcttgacagagacacagtttttgctctggtgaattacatcttctttaaaggcaaatgggagagaccctttgaagtcaaggacaccgaggaagaggacttccacgtggaccaggtgaccaccgtgaaggtgcctatgatgaagcgtttaggcatgtttaacatccagcactgtaagaagctgtccagctgggtgctgctgatgaaatacctgggcaatgccaccgccatcttcttcctgcctgatgaggggaaactacagcacctggaaaatgaactcacccacgatatcatcaccaagttcctggaaaatgaagacagaaggtctgccagcttacatttacccaaactgtccattactggaacctatgatctgaagagcgtcctgggtcaactgggcatcactaaggtcttcagcaatggggctgacctctccggggtcacagaggaggcacccctgaagctctccaaggccgtgcataaggctgtgctgaccatcgacgagaaagggactgaagctgctggggccatgtttttagaggccatacccatgtctatcccccccgaggtcaagttcaacaaaccctttgtcttcttaatgattgaacaaaataccaagtctcccctcttcatgggaaaagtggtgaatcccacccaaaaataactgcctctcgctcctcaacccctcccctccatccctggccccctccctggatgacattaaagaagggttgagctggtccctgcctgcatgtgactgtaaatccctcccatgttttctctgagtctccctttgcctgctgaggctgtatgtgggctccaggtaacagtgctgtcttcgggccccctgaactgtgttcatggagcatctggctgggtaggcacatgctgggcttgaatccaggggggactgaatcctcagcttacggacctgggcccatctgtttctggagggctccagtcttccttgtcctgtcttggagtccccaagaaggaatcacaggggaggaaccagataccagccatgaccccaggctccaccaagcatcttcatgtccccctgctcatcccccactcccccccacccagagttgctcatcctgccagggctggctgtgcccaccccaaggctgccctcctgggggccccagaactgcctgatcgtgccgtggcccagttttgtggcatctgcagcaacacaagagagaggacaatgtcctcctcttgacccgctgtcacctaaccagactcgggccctgcacctctcaggcacttctggaaaatgactgaggcagattcttcctgaagcccattctccatggggcaacaaggacacctattctgtccttgtccttccatcgctgccccagaaagcctcacatatctccgtttagaatcaggtcccttctccccagatgaagaggagggtctctgctttgttttctctatctcctcctcagacttgaccaggcccagcaggccccagaagaccattaccctatatcccttctcctccctagtcacatggccataggcctgctgatggctcaggaaggccattgcaaggactcctcagctatgggagaggaagcacatcacccattgacccccgcaacccctccctttcctcctctgagtcccgactggggccacatgcagcctgacttctttgtgcctgttgctgtccctgcagtcttcagagggccaccgcagctccagtgccacggcaggaggctgttcctgaatagcccctgtggtaagggccaggagagtccttccatcctccaaggccctgctaaaggacacagcagccaggaagtcccctgggcccctagctgaaggacagcctgctccctccgtctctaccaggaatggccttgtcctatggaaggcactgccccatcccaaactaatctaggaatcactgtctaaccactcactgtcatgaatgtgtacttaaaggatgaggttgagtcataccaaatagtgatttcgatagttcaaaatggtgaaattagcaattctacatgattcagtctaatcaatggataccgactgtttcccacacaagtctcctgttctcttaagcttactcactgacagcctttcactctccacaaatacattaaagatatggccatcaccaagccccctaggatgacaccagacctgagagtctgaagacctggatccaagttctgacttttccccctgacagctgtgtgaccttcgtgaagtcgccaaacctctctgagccccagtcattgctagtaagacctgcctttgagttggtatgatgttcaagttagataacaaaatgtttatacccattagaacagagaataaatagaactacatttcttgca

(19) United States
(12) Patent Application Publication
(10) Pub. No.: US 2025/0257403 A1
(43) Pub. Date: Aug. 14, 2025

(54) METHOD TO DETECT ABNORMAL ALPHA-1 ANTITRYPSIN DEFICIENCY (AATD) GENOTYPES

(71) Applicant: NATIONAL JEWISH HEALTH, Denver, CO (US)

(72) Inventors: Yongbao WANG, Denver, CO (US); Iara MACHADO, Denver, CO (US); Ron HARBECK, Denver, CO (US); Robert SANDHAUS, Denver, CO (US); Claire COESHOTT, Denver, CO (US)

(21) Appl. No.: 18/864,402

(22) PCT Filed: May 12, 2023
(86) PCT No.: PCT/US2023/066932

§ 371 Date: Nov. 8, 2024

Related U.S. Application Data
(60) Provisional application No. 63/341,300, filed on May 12, 2022.

(51) Int. Cl. C12Q 1/6883; C12Q 1/686; C12Q 1/6872

(52) U.S. Cl. CPC C12Q 1/6883; C12Q 1/686; C12Q 1/6872; C12Q 2600/156

(57) ABSTRACT

Disclosed herein are methods and kits for detecting abnormal alpha-1 antitrypsin deficiency (AATD) genotypes.

### CROSS REFERENCE TO RELATED APPLICATIONS

This application claims the benefit of priority under 35 U.S.C. § 119(e) to U.S. Provisional Patent Application No. 63/341,300, filed May 12, 2022. The entire disclosure of U.S. Provisional Patent Application No. 63/341,300 is incorporated herein by reference.

### REFERENCE TO SEQUENCE LISTING

This application contains a Sequence Listing submitted as an electronic XML file named “2879-239-PCT.xml”, having a size of 11,000 bytes, and created on May 12, 2023. The information contained in this electronic XML file is hereby incorporated by reference in its entirety pursuant to 37 CFR § 1.52(e)(5).

### BACKGROUND

Alpha-1 antitrypsin deficiency (AATD) is a common, but under-diagnosed, inherited disease caused by specific serpin family A member 1 (SERPINA1) gene mutations. Genetic changes can lead to low or abnormal alpha-1 antitrypsin protein (AAT, also referred to as A1AT) production in liver cells, with resultant liver or lung damage. Current clinical diagnostic tests include measuring AAT protein levels, detecting deficient protein alleles by isoelectric focusing gel (IEF), genetic polymerase chain reaction (PCR) testing for major S and Z allele mutations as well as the normal allele, M, and whole gene sequencing. IEF is a labor-intensive assay and results can be hard to interpret and most PCR based genotyping assays only detect two major single nucleotide polymorphism (SNPs). Thus, there is a need for a more accessible genetic front-line test to cover more mutations which allows for broader screening for lung and liver diseases in patients.

### SUMMARY

One embodiment relates to a method to detect an abnormal alpha-1 antitrypsin deficiency (AATD) genotype in a subject comprising: obtaining a biological sample from the subject; detecting the presence of one or more single nucleotide polymorphism (SNP) specific oligonucleotides in serpin family A member 1 (SERPINA1) protein in DNA from the biological sample comprising: performing a first polymerase chain reaction (PCR) reaction comprising specific SERPINA1 primers producing a first PCR product; performing a single SNP-specific primer extension PCR reaction comprising specific SERPINA1 extension primers with the first PCR product producing an extended PCR product; and performing matrix assisted laser desorption ionization time-of-flight mass spectrometry (MALDI-TOF MS) on the extended PCR product to detect the SNP-specific oligonucleotide; analyzing the genotype of the detected one or more SNPs; and determining the subject has an abnormal AATD genotype if the one or more SNPs is detected in the sample and is determined not to comprise a normal genotype for AATD.

In one aspect, the subject has a disease or condition selected from the group consisting of emphysema, chronic obstructive pulmonary disease (COPD), asthma, and necrotizing panniculitis.

In still another aspect, the subject is an AATD asymptomatic subject with persistent obstruction on pulmonary function testing having risk factors selected from the group consisting of cigarette smoking and occupational exposure.

In another aspect, the subject is an AATD asymptomatic subject having a family history of AATD.

In yet another aspect, the subject is a newborn.

Another embodiment relates to a kit for detecting an abnormal alpha-1 antitrypsin deficiency (AATD) genotype comprising: (a) at least one primer set capable of detecting SERPINA1 in a biological sample; (b) at least one extension primer set capable of detecting at least one single SNP in SERPINA1 in a biological sample; and an instruction for using the primer set of (a) and the extension primer set of (b), wherein the instruction provides guidance to use the primer set and the extension primer set in performing PCR in detecting SNP-specific oligonucleotides in a biological sample.

In any of the embodiments disclosed herein, the biological sample is selected from the group consisting of whole blood, nasal mucosa and buccal mucosa.

In any of the embodiments disclosed herein, the SNPs that are detected comprise rs6647, rs764325655, rs28929470, rs28931570, rs121912713, rs28931569, rs121912714, rs775982338, rs17580, rs28931568, rs28929474, rs11558261, rs199422209, rs199422211, rs28931572, rs751235320, rs55819880, rs267606950, rs28929473, rs1057516212 or combinations thereof.

### DETAILED DESCRIPTION

This invention generally relates to methods for detecting abnormal AATD genotypes.

The assay disclosed herein is based on PCR followed by a single strand primer-specific extension PCR reaction after shrimp alkaline phosphatase (SAP) cleanup of the first PCR product to generate single nucleotide polymorphisms (SNP) specific oligonucleotides. The detection of these oligo products is performed on matrix assisted laser desorption ionization time-of-flight mass spectrometry (MALDI-TOF MS). The PCR and extension primers were designed to target specifically the SNPs listed in Table 1, including common S, Z, less common F and I and other uncommon SNPs. A total of twenty SNPs were multiplexed within one reaction. Each SNP was differentiated by extended oligo products with defined molecular weights between 4000 to 8000 Daltons. In Table 1, “PI typing” stands for “protease inhibitor” typing; “dbSNP ID” stands for “database SNP identification” at the National Center for Biotechnology Information (NCBI); the nucleotide change column provides the NM accession number which links to the mRNA record at the NCBI database and indicates the nucleotide change; the “amino acid change” column provides the specific amino acid change and location (i.e. amino acid position number) of the amino acid change within the sequence. As provided for herein the protein sequence for alpha-1-antitrypsin having NCBI reference number NP_000286.3 is SEQ ID NO:1. The nucleotide sequence for NCBI reference number NM_000295.5 is SEQ ID NO:2 and the nucleotide sequence for NCBI reference number NM_001127701.2 is SEQ ID NO:3.

TABLE 1
SNPs detected by assay

| PI typing | dbSNP ID | Nucleotide change | Amino Acid change |
|---|---|---|---|
| M | rs6647 | NM_001127701. 1 c.710T > C | p.Val237Ala |
| Q0clayton | rs764325655 | NM_000295.5 c.1158dup | p.Glu387fsTer14 |
| F | rs28929470 | NM_001127701. 1 c.739C > T | p.Arg247Cys |
| I | rs28931570 | NM_001127701. 1 c.187C > T | p.Arg63Cys |
| Pittsburg | rs121912713 | NM_001127701. 1 c.1145T > G | p.Met382Arg |
| Mprocida | rs28931569 | NM_001127701. 1 c.194T > C | p.Leu65Pro |
| Mplowell | rs121912714 | NM_001127701. 1 c.839A > T | p.Asp280Val |
| Mmalton | rs775982338 | NM_000295.5 c.227_229delTCT | p.Phe76del |
| S | rs17580 | NM_001127701. 1 c.863A > T | p.Glu288Val |
| Mmineral Springs | rs28931568 | NM_001127701. 1 c.272G > A | p.Gly91Glu |
| Z | rs28929474 | NM_001127701. 1 c.1096G > A | p.Glu366Lys |
| Q0newport | rs11558261 | NM_001127701. 1 c.415G > A | p.Gly139Ser |
| Qheerlen | rs199422209 | NM_001127701. 1 c.1178C > T | p.Pro393Leu |
| Q0bellingham | rs199422211 | NM_000295.5 c.721A > T | p.Lys241Ter |
| Q0ludwigshafen | rs28931572 | NM_001127701. 1 c.347T > A | p.Ile116Asn |
| Q0west | rs751235320 | NM_000295.5 c.646 + 1G > T | Protein absence |
| Siiyama | rs55819880 | NM_001127701. 1 c.230C > T | p.Ser77Phe |
| Q0granite Falls | rs267606950 | NM_000295.5 c.552del | p.Asp183_Tyr184delinsTer |
| Q0matawa | rs28929473 | NM_001127701. 1 c.1131A > T | p.Leu377Phe |
| Q0casablanca | rs1057516212 | NM_000295.5 c.288_291del | p.His97fs |

The present inventors have made the surprising discovery that the multiplex genotyping assay disclosed herein accurately detected 20 SNPs (Table 1) and was more accurate than IEF. With the ability to run 96 or 384 samples per run, this assay can be used for a broad AATD screening and diagnosis, especially in people with symptoms of hepatic or lung disorders at any age when there is no obvious cause. The diagnosis can thus also lead to treatment of the subject.

As disclosed herein, a subject having a normal AATD genotype means that only the M allele (or only the M SNIP and none of the other SNPs in Table 1) is detected in a single sample by the assays disclosed herein. A subject having a normal AATD genotype does not have alpha-1 antitrypsin deficiency (AATD) and is a subject with normal alpha-1 antitrypsin proteins (referred to as Pi*MM). The SNIP in Table 1 for M is rs6647.

As disclosed herein, a subject having an abnormal AATD genotype means that the genotype detected or determined by the assay disclosed herein contains at least one SNP from table 1 that is not M. Examples of abnormal AATD genotypes include but are not limited to: where “M” is one allele and the second allele is any other non-M allele in Table 1; where “F” is one allele and the second allele is “F” or any other allele in Table 1; where “S” is one allele and the second allele is “S” or any other allele in Table 1; where “Z” is one allele and the second allele is “Z” or any other allele in Table 1; where “QOclayton” is one allele and the second allele is “QOclayton”; where one allele is “Mprocida” and the second allele is “Mprocida”, where one allele to “Mplowell” and the second allele is “Mplowell”; where one allele is “Mmalton” and the second allele is “Mmalton”, where one allele is “Mmineral Springs” and the second allele is “Mmineral Springs”; where one allele is “QOnewport” and the second allele is “QOnewport”; where one allele is “Qheerlen” and the second allele is “Qheerlen”; where one allele is “QObellingham” and the second allele is “QObellingham”; where one allele is “QOludwigshafen” and the second allele is “QOludwigshafen”; where in one allele is “QOwest” and the second allele is “QOwest”; where one allele is “Siiyama” and the second allele is “Siiyama”; where one allele is “QOgranite Falls” and the second allele is “QOgranite Falls”; where in one allele is “QOmatawa” and the second allele is “QOmatawa”; and where one allele is “QOcasablanca” and the second allele is “QOcasablanca”.

One embodiment is to determine and/or detect an abnormal alpha-1 antitrypsin deficiency (AATD) genotype in a subject. This assay includes obtaining a biological sample from the subject; detecting the presence of one or more single nucleotide polymorphism (SNP) specific oligonucleotides in serpin family A member 1 (SERPINA1) protein in DNA from the sample. This step comprises performing a first PCR reaction comprising specific SERPINA1 primers producing a first PCR product; then performing a single SNP-specific primer extension PCR reaction comprising specific SERPINA1 extension primers with the first PCR product producing an extended PCR product; and performing matrix assisted laser desorption ionization time-of-flight mass spectrometry (MALDI-TOF MS) on the extended PCR product to detect the SNP-specific oligonucleotides. analyzing the genotype of the detected one or more SNPs; determining the subject has an abnormal AATD genotype if the one or more SNPs is detected in the sample and is determined not to comprise a normal genotype for AATD.

Another embodiment relates to a method to genotype AATD in a subject comprising: obtaining a biological sample from the subject; detecting and genotyping SNP specific oligonucleotides in DNA from the biological sample comprising: performing a first PCR reaction comprising SERPINA1 primers producing a first PCR product; performing a single SNP-specific primer extension PCR reaction comprising specific SERPINA1 extension primers with the first PCR product producing an extended PCR product; and performing MALDI-TOF MS on the extended PCR product to detect and genotype the SNP-specific oligonucleotides.

The subject can have a disease or condition selected from the group consisting of emphysema, chronic obstructive pulmonary disease (COPD), asthma, and necrotizing panniculitis. Further, the subject can be an AATD asymptomatic subject with persistent obstruction on pulmonary function testing having risk factors selected from the group consisting of cigarette smoking and occupational exposure. Further, in one aspect, the subject is an AATD asymptomatic subject having a family history of AATD. In one aspect, the subject is a newborn. The subject may be viewed as a patient. The subject can be a human, and the human can be a patient. The terms, “subject,” “patient,” and “individual” are used interchangeably and refer to either a human or a non-human animal. These terms include mammals such as humans, primates, livestock animals e bovines, porcines), companion animals (e.g., canines, felines) and rodents (e.g., mice, rabbits and rats).

The sample can be a sample taken from, or provided by, a subject. Samples may include, but are not limited to, body fluids (e.g. whole blood, dry blood, isolated cells from blood, plasma, serum, cerebrospinal fluid, GI tract contents, semen, urine, stool/fecal); tissues (e.g. nasal mucosa, buccal mucosa, adrenal, hepatic, renal, pancreatic, pituitary, thyroid, immune, ovarian, testicular, prostate, endometrial, ocular, mammary, adipose, epithelial, endothelial, neural, muscle, pulmonary, epidermis, and osseous) as well as samples obtained by e.g. a swab, rinse or scrape of a tissue, body cavity or biopsy.

The term “sample” also encompasses swabs of the abovementioned samples. In the clinical setting, swabs are primarily used to diagnose clinical diseases. The term “sample” encompasses functional equivalents of swabs, regardless of the functional equivalents' sensitivity or reproducibility relative to swabs. In addition to swabs, tissues, cotton balls/pads, wipes, are also encompassed by the term “sample”.

The method relates to obtaining DNA from a sample, amplifying a desired region of the DNA, detecting signature characteristics of the amplified region, and identifying the specific SNPs in the sample based on those signature characteristics. First, a sample is obtained from any of a variety of sources, including but not limited to whole blood, dried blood or buccal swabs from tissue. DNA is typically then extracted from the sample, however, the sample can also be used without having first extracted the DNA. One or more regions suspected of being present in the sample is then amplified using primers that anneal to sequences that are specific for the SNPs in Table 1. The amplified regions are then used in PCR based extension reactions. The extension reactions involve using a primer, wherein the primer can be specific to one or more SNPs, to produce an extension reaction product from the amplified region. The extension reaction product will have signature characteristics that can be used to identify and distinguish types of SNPs. The extension product is then analyzed to detect the specific-SNP-specific oligonucleotides in the sample. In some embodiments, the extension reaction product is analyzed to determine its molecular weight and/or mass to charge ratio.

DNA isolation from blood, plasma, or serum can be performed using any method known to one skilled in the art. One such method is disclosed in Chiu, R. W. K. et al. Clin Chem 47:1607-1613. (2001) incorporated herein by reference in its entirety. Other suitable methods include, for example TRI REAGENT® BD (Molecular Research Center, Inc., Cincinnati, Ohio), which is a reagent for isolation of DNA from, for example, plasma. TRI REAGENT BD and the single-step method are described, for example, in the U.S. Pat. Nos. 4,843,155 and 5,346,994.

Protein levels in the biological sample can be determined by nephelometry.

Polymerase chain reaction, or PCR, is a technique described in Molecular Cloning, A Laboratory Manual, Second Edition, Cold Spring Harbor Laboratory Press (1989) and Current Protocols in Molecular Biology, Supplement 1, John Wiley & Sons (1987-1997). Many variations of PCR have been developed, for instance, real time PCR (also known as quantitative PCR or qPCR), hot-start PCR, competitive PCR, and others, and these may all be employed where appropriate.

In some embodiments, using a PCR based amplification, the oligonucleotide primers of the invention are contacted with a reaction mixture containing a target sequence and free nucleotides in a suitable buffer. Thermal cycling of the resulting mixture in the presence of a DNA polymerase results in amplification of the sequence between the primers.

Due to the specificity of the base extension reaction, detection of the SNPs disclosed herein can be accurately reproduced for analysis. This includes differences ranging from single nucleotide variations to small and large deletions, insertions, substitutions, and other types of nucleic acid variations present in even a small percentage of the pool of nucleic acids present in a sample.

The base extension reaction according to the present invention can be performed using any appropriate base extension method. In general, a nucleic acid extension primer is designed to anneal to the target nucleic acid. One or more extension primers of the invention are contacted with a reaction mixture suspected of containing the target sequence, in the presence of free nucleotides, a polymerase enzyme, and any other desired components in a suitable buffer. The extension reaction is then carried out using parameters, such as temperature and duration, that allow synthesis, primed by the extension primer, of an extension reaction product from the corresponding template target sequence.

As with PCR, the parameters used for extension reactions can be adjusted based on the particular components of the reaction, for example for the type of polymerase and the extension primers. However, unlike PCR, extension reactions do not involve thermal cycling to amplify, as opposed to producing a single copy of, a sequence. That is, extension reactions involve only one cycle and produce a single copy of the desired sequence.

The oligonucleotide primers of the present invention can comprise up to 100 nucleotides, preferably up to 80, 60, 50, 40, 30, 25, 24, 23, 22, 21, 20, 19, 18, 17, 16, 15, 14, 13, 12, 11, 10, 9, 8, 7, or 6 nucleotides. The oligonucleotide primers may comprise at least 7, 8, 9, 10, 11, 12, 13, 14, 15, 16, 17, 18, 19, 20, 21, 22, 23, 24, 25, 30, 40, 60, or at least 80 nucleotides. The nucleotides of the oligonucleotide can be any type of nucleotide so long as hybridization specificity or efficiency and amplification efficiency is not detrimentally affected. The oligonucleotide may therefore be a deoxyribonucleotide, a ribonucleotide, modifications thereof (e.g. PNA, morpholino-, LNA) and mixtures thereof DNA oligonucleotides and LNA modified DNA oligonucleotides are preferred.

In some embodiments, the extension reaction products are analyzed by mass spectrometry. The type of mass spectrometry can be any suitable type, including matrix assisted laser desorption ionization (MALDI), electrospray (ES), ion cyclotron resonance (ICR) and Fourier Transform.

With MALDI mass spectrometry, various mass analyzers can be used, e.g., magnetic sector/magnetic deflection instruments in single or triple quadrupole mode (MS/MS), Fourier transform and time-of-flight (TOF) configurations as is known in the art of mass spectrometry. For the desorption/ionization process, numerous matrix/laser combinations can be used. Ion-trap and reflectron configurations can also be employed. In one embodiment of the present invention, MALDI-TOF is employed to analyze the extension reaction products.

The methods of the invention are amenable to multiplexing, or the testing of a sample simultaneously. Consistent with the amplification and extension reactions described above, multiple different primers can be used in a single reaction step. That is, more than one primer pair or more than two primers can be used in the amplification reaction, and more than one primer can be used in the extension reaction.

The present invention also provides kits that include at least one component to assist one in detecting abnormal AATD genotypes. By way of non-limiting example, kits may include at least one SNP detection reagent, such as a primer set (i.e., forward primers and reverse primers that target against amplification near the SNP regions of the SERPINA1). For example, kits are provided for identifying abnormal AATD genotypes. Example kits may include at least one SERPINA primer set containing a forward primer and a reverse primer that are capable of being used to detect a first PCR product. Such a kit further includes at least one extension primer set to produce an extended PCR product and contains a forward primer and a reverse primer that are capable of being used to detect at least one SNP in Table 1. The instructions may include instructions for using the primers in performing PCR in detecting in detecting abnormal AATD genotypes in a biological sample.

The instructions present in such a kit may instruct the user for example, on how to use the components of the kit to perform the various methods provided herein. In particular, kits may include instructions for detecting SNPs using primer sets.

Articles of manufacture and kits provided herein may addi-tionally include reagents for carrying out the methods disclosed herein (e.g., buffers, Taq polymerase enzymes, co-factors, and agents to prevent contamination). Such reagents may be specific for one of the commercially available instruments described herein.

The term “pharmaceutically acceptable carrier” refers to a non-toxic carrier that may be administered to a patient, together with compositions of this invention, and which does not destroy the pharmacological activity of the active agents within the composition. The term “excipient” refers to an additive in a formulation or composition that is not a pharmaceutically active ingredient.

The term “pharmaceutically effective amount” refers to an amount effective to treat a patient, e.g., effecting a beneficial and/or desirable alteration in the general health of a patient suffering from a disease (including but not limited to cancer or viral or other infection). A “pharmaceutically effective amount” also refers to the amount required to improve the clinical symptoms of a patient.

Throughout this specification, the word “comprise” or variations such as “comprises” or “comprising” will be understood to imply the inclusion of a stated integer (or components) or group of integers (or components), but not the exclusion of any other integer (or components) or group of integers (or components).

The singular forms “a,” “an,” and “the” include the plurals unless the context clearly dictates otherwise.

The term “including” is used to mean “including but not limited to.” “Including” and “including but not limited to” are used interchangeably.

The term “mammal” refers to organisms from the taxonomy class “mammalian,” including but not limited to humans, other primates such as chimpanzees, apes, orangutans and monkeys, rats, mice, cats, dogs, cows, horses, etc.

Unless otherwise specified, it is to be understood that each embodiment of the invention may be used alone or in combination with any one or more other embodiments of the invention.

Those skilled in the art will recognize or be able to ascertain using no more than routine experimentation, numerous equivalents to the compounds, compositions, and methods of use thereof described herein. Such equivalents are considered to be within the scope of the invention.

The contents of all references, patents and published patent applications cited throughout this Application, as well as their associated figures are hereby incorporated by reference in their entirety. In case of conflict, the present specification, including its specific definitions, will control.

The following experimental results are provided for purposes of illustration and are not intended to limit the scope of the invention.

### EXAMPLES

### Example 1. Cohort Study with Biobank Samples

Methods: Blood or buffy coat samples (177), archived in National Jewish Health Biobank with AATD indication, and 176 presumed normal MM (i.e., having MM genotype-see Table 1; PI*MM or MM) samples were tested. PCR was followed by single SNP-specific primer extension, then SNP-specific extended oligonucleotides were detected by Matrix-Assisted Laser desorption/ionization time-of-flight (MALDI-TOF). Extension primers were designed to include phenotypes:
PI*M, PI*S, PI*Z, PI*F, PI*I, PI*Pittsburgh, PI*Mheerlen, PI*Mmalton, PI*Mmineral spring, PI*Mprocida, PI*Lowell, PI*QOclayton, PI*QOBellingham, PI*QOgranite falls, PI*QOLudwigshafen, PI*QOmatawa, PI*QOwest, PI*Siiyama, PI*Newport and H97* (also referred to as PI*QOcasablanca).
   Twenty SNPs were multiplexed within one reaction. Sanger sequencing was performed to confirm each genotype and to confirm samples with discordant results between phenotyping (IEF) and genotyping by the assay.

Results: The assay detected all 20 SNPs (see Table 1) to a minimum of 0.5 ng/μl DNA with 100% precision. Sample types include whole blood, dry blood spot and buccal swabs. 89.5% (77/86) agreement was achieved between this assay and historical IEF for samples with known phenotypes. Sanger sequencing confirmed that DNA sequences from 9 discordant samples were in agreement with results from this assay. (Table 2)

TABLE 2
Genotyping results from known phenotyped samples and
discordant samples were tested by Sanger sequencing.

| Genotype | MS | MZ | ZZ | MI | SZ | MF | FZ | MM | Total |
|---|---|---|---|---|---|---|---|---|---|
| Sample # | 31 | 39 | 5 | 1 | 5 | 1 | 1 | 3 | 86 |
| Sample % | 36.0% | 45.3% | 5.8% | 1.2% | 5.8% | 1.2% | 1.2% | 3.5% | |

| | SAMPLE ID | | | | | | | | |
|---|---|---|---|---|---|---|---|---|---|
| Assays | BIO412 | BIO574 | BIO277 | BIO914 | BIO729 | BIO330 | BIO840 | BIO520 | BIO972 |
| PI from | MS | ZZ | SS | MZ | SS | M | MS | SS | MZ |
| Biobank | | | | | | | | | |
| (IEF) | | | | | | | | | |
| Genotype, | MM | MZ | MS | MM | MS | MS | SZ | MS | ZZ |
| this assay | | | | | | | | | |
| Sanger | MM | MZ | MS | \hat{ }M, | MS | MS | SZ | MS | ZZ |
| sequencing | | | | rs70993 | | | | | |
| *level | 152 | 79/97/89 | 143 | 75 | 102 | 121 | 75 | 100 | 136 |
| (Biobank), | | | | | | | | | |
| mg/dL | | | | | | | | | |

*Normal MM level: 104-276 mg/dL

SNPs were determined for 95.6% (87) of 91 samples from the biobank with unknown phenotype but with AATD indication. Four samples with uncommon genotypes (MMplowell, Mmalton, ZI and MI) and the four samples with MM were confirmed by Sanger sequencing. (Table 3).

TABLE 3
Genotyping results from samples with unknown phenotypes and rare
and normal genotypes were confirmed by Sanger sequencing.

| Genotype | MS | MZ | ZZ | MI | SZ | MF | ZI | MMplowell | MmaltonZ | MM | Total |
|---|---|---|---|---|---|---|---|---|---|---|---|
| Sample # | 22 | 34 | 16 | 1 | 8 | 3 | 1 | 1 | 1 | 4 | 91 |
| Sample % | 24.2 | 37.4 | 17.6 | 1.1 | 8.8 | 3.3 | 1.1 | 1.1 | 1.1 | 4.4 | |

| Assays | SAMPLE ID | | | | | | | |
|---|---|---|---|---|---|---|---|---|
| (BIO#) | BIO59 | BIO133 | BIO141 | BIO93 | BIO91 | BIO101 | BIO102 | BIO124 |
| Genotype, | MI | ZI | MMplowell | MmaltonZ | MM | MM | MM | MM |
| this assay | | | | | | | | |
| Sanger | MI | ZI | MMplowell | MmaltonZ | MM | MM | MM | MM |
| sequencing | | | | | | | | |
| *level | 138 | 74/97 | 85 | <30 | 139/172 | N/A | 128 | 209 |
| (Biobank), | | | | | | | | |
| mg/dL | | | | | | | | |

*Normal MM level: 104-276 mg/dL

From a total cohort of 177 biobank samples, 96.600 (171) were genotyped as abnormal SNPs and confirmed by Sanger sequencing with representation for each genotype. For 176 presumed normal MMN samples, 91% (17) were genotyped as MS, MW, MF, FF or MI (Table 4). All others were genotyped as normal MM. F allele accounts for 1.1% of the group and one FF genotype was identified by this assay and confirmed by Sanger sequencing.

TABLE 4
Summary of presumed normal MM samples.

| | | | | | | | Total |
|---|---|---|---|---|---|---|---|
| TOTAL | FF | MF | MI | MS | MZ | MM | Abnormal |
| 176 | 1 | 2 | 1 | 9 | 3 | 160 | 16 |
| 100% | 0.6% | 1.1% | 0.6% | 5.1% | 1.7% | 90.9% | 9.1% |

While various embodiments of the present invention have been described in detail, it is apparent that modifications and adaptations of those embodiments will occur to those skilled in the art. It is to be expressly understood, however, that such modifications and adaptations are within the scope of the present invention, as set forth in the following exemplary claims.

What is claimed:

1. A method to detect an abnormal alpha-1 antitrypsin deficiency (AATD) genotype in a subject comprising:
a. obtaining a biological sample from the subject;
b. detecting the presence of one or more single nucleotide polymorphism (SNP) specific oligonucleotides in serpin family A member 1 (SERPINA1) protein in DNA from the biological sample comprising:
1. performing a first polymerase chain reaction (PCR) reaction comprising specific SERPINA1 primers producing a first PCR product;
2. performing a single SNP-specific primer extension PCR reaction comprising specific SERPINA1 extension primers with the first PCR product producing an extended PCR product; and
3. performing matrix assisted laser desorption ionization time-of-flight mass spectrometry (MALDI-TOF MS) on the extended PCR product to detect the SNP-specific oligonucleotide;
c. analyzing the genotype of the detected one or more SNPs;
d. determining the subject has an abnormal AATD genotype if the one or more SNPs is detected in the sample and is determined not to comprise a normal genotype for AATD.

2. The method of claim 1, wherein the subject has a disease or condition selected from the group consisting of emphysema, chronic obstructive pulmonary disease (COPD), asthma, and necrotizing panniculitis.

3. The method of claim 1, wherein the subject is an asymptomatic subject with persistent obstruction on pulmonary function testing having risk factors selected from the group consisting of cigarette smoking and occupational exposure.

4. The method of claim 1, wherein the subject is an AATD asymptomatic subject having a family history of AATD.

5. The method of claim 1, wherein in the subject is a newborn.

6. The method of claim 1, wherein the biological sample is selected from the group consisting of whole blood, dry blood, isolated cells from blood, nasal mucosa and buccal mucosa.

7. The method of claim 1, wherein the SNPs comprise rs6647, rs764325655, rs28929470, rs28931570, rs121912713, rs28931569, rs121912714, rs775982338, rs17580, rs28931568, rs28929474, rs11558261, rs199422209, rs199422211, rs28931572, rs751235320, rs55819880, rs267606950, rs28929473, rs1057516212 or a combination thereof.

8. A kit for detecting an abnormal alpha-1 antitrypsin deficiency (AATD) genotype comprising:
a. at least one primer set capable of detecting SERPINA1 in a biological sample;
b. at least one extension primer set capable of detecting at least one single SNP in SERPINA1 in a biological sample; and
c. an instruction for using the primer set of (a) and the extension primer set of (b), wherein the instruction provides guidance to use the primer set and the extension primer set in performing PCR in detecting SNP-specific oligonucleotides in a biological sample.

9. The kit of claim 8, wherein the biological sample is selected from the group consisting of whole blood, dry blood, isolated cells from blood, nasal mucosa and buccal mucosa.

10. The kit of claim 8, wherein the SNPs comprise rs6647, rs764325655, rs28929470, rs28931570, rs121912713, rs28931569, rs121912714, rs775982338, rs17580, rs28931568, rs28929474, rs11558261, rs199422209, rs199422211, rs28931572, rs751235320, rs55819880, rs267606950, rs28929473, rs1057516212 or a combination thereof.
